(19) United States
(12) Patent Application Publication
(10) Pub. No.: US 2024/0049982 A1
(43) Pub. Date: Feb. 15, 2024

(54) Estimation of Individual's Maximum Oxygen Uptake, VO2MAX

(71) Applicant: Huawei Technologies Co., Ltd., Shenzhen (CN)

(72) Inventor: Mario Costa, Kista (SE)

(21) Appl. No.: 18/260,142

(22) PCT Filed: Dec. 30, 2020
(86) PCT No.: PCT/EP2020/088023

§ 371 Date: Jun. 30, 2023

(51) Int. Cl. A61B 5/083; A61B 5/024; A61B 5/0205; A61B 5/00; A63B 24/00; G16H 50/30; G16H 40/67

(52) U.S. Cl. CPC A61B 5/0833; A61B 5/02438; A61B 5/0205; A61B 5/7278; A63B 24/0062; G16H 50/30; G16H 40/67

(57) ABSTRACT

A wearable device to estimate an individual's maximum oxygen uptake (VO2max) during exercise. The wearable device includes a processor and a memory. The processor is configured to receive heart-rate measurement data and exercise workload data for an individual of the wearable device. The memory stores instructions that cause the processor to obtain the heart-rate and exercise workload of the individual, normalize the heart-rate with respect to the individual's maximum heart-rate to provide a data pair of normalized heart-rate (HRn) and exercise workload (w), and apply a probabilistic model that relates HRn to w and maximum oxygen uptake to provide an estimate of maximum oxygen uptake (VO2max) of the individual.

### TECHNICAL FIELD

The disclosure relates to a method for estimating an individual's maximum oxygen uptake, during exercise, a wearable device and a computing device for estimating an individual's maximum oxygen uptake, VO2max.

### BACKGROUND

Cardiovascular fitness is important in the fields of sports, fitness, diagnostic, prognostic, and self-monitoring in asymptomatic individuals. Direct measurement of fitness requires a maximal exercise test and takes place in a laboratory, which is associated with an increased risk of cardiovascular events. Indirect estimation of fitness overcomes some of the limitations of the direct measurement, but it still requires an individual to undergo rigorously a different kind of fixed test protocol. Maximal oxygen uptake or consumption (VO2max) of an individual means the maximal capacity of the individual to perform aerobic work. In general, the maximum oxygen uptake (VO2max) refers to a maximum rate of oxygen consumption measured during an exercise of increasing intensity. The measurement of VO2max provides a quantitative value of endurance fitness for comparison of individual training effects and between people in endurance training. Maximal oxygen consumption reflects cardiorespiratory fitness and endurance capacity during aerobic exercise.

In known approaches, the maximum oxygen uptake (VO2max) quantity is determined without requiring any maximal effort and these procedures may be based on freely performed everyday exercise of the individual. The analytics for determining the maximum oxygen uptake (VO2max) are based on detecting heart-rate responses during each recorded workout (such as running, walking or cycling) that help to determine changes in fitness levels. These changes are used to adapt the training program and optimize training loads for faster progress. A known maximum oxygen uptake (VO2max) estimation system employs an algorithm that analyzes the relationship between heart-rate and running speed at multiple points during a training (running) session. However, it requires a user to run at multiple different speeds.

Therefore, there arises a need to address the aforementioned technical drawbacks in existing systems or technologies in the estimation of maximum oxygen uptake (VO2max).

### SUMMARY

It is an object of the disclosure to provide an improved method for estimating an individual's maximum oxygen uptake, VO2max, during exercise while avoiding one or more disadvantages of prior art approaches.

This object is achieved by the features of the independent claims. Further implementations are apparent from the dependent claims, the description, and the figures.

The disclosure provides a method, a wearable device, and a computing device for estimating an individual's maximum oxygen uptake, VO2max, during exercise.

According to a first aspect, there is provided a method of estimating an individual's maximum oxygen uptake, VO2max, during exercise, the individual having a heart rate, the method comprising:
obtaining the heart-rate and exercise workload of the individual;
normalising the obtained heart-rate with respect to the individual's maximum heart-rate to provide a data pair of normalised heart-rate, HRn, and exercise workload, w; and
applying a probabilistic model that relates normalised heart-rate to exercise workload and maximum oxygen uptake to provide an estimate of the individual's maximum oxygen uptake, VO2max.

The advantage of the method is that it can estimate an individual's maximum oxygen uptake, VO2max, from for example free-running exercises performed at a single running speed. Additionally, the method can further improve the VO2max estimation by using data from multiple exercise sessions.

The estimate of the individual's maximum oxygen uptake, VO2max, may be provided by using the probabilistic model to determine a probability density function p(VO2max|HRn, w). The probability density function p(VO2max|HRn, w) may be determined using Bayes' Rule:

$$p ( VO 2 max \vert HRn , w ) = \frac{p ( HRn , w \vert VO 2 max ) p ( VO 2 max )}{p ( HRn , w )}$$

According to a second aspect, there is provided a wearable device to estimate an individual's maximum oxygen uptake, VO2max, during exercise, the wearable device comprising a processor and being configured to receive heart-rate measurement data and exercise workload data for an individual of the device; and a memory storing instructions that cause the processor to perform the above method.

The wearable device optionally includes a wireless interface for receiving the heart-rate measurement data and the exercise workload data from one or more sensing arrangements external to the wearable device.

The advantage of the wearable device is that it can estimate an individual's maximum oxygen uptake, VO2max, without the requirement that exercise be performed at different intensities. Additionally, the wearable device can improve VO2max estimation by using data from multiple exercise sessions.

According to a third aspect, there is provided a computing device to estimate an individual's maximum oxygen uptake, VO2max, during exercise, the computing device comprising a processor, a communication interface coupled to the processor to receive heart-rate measurement data and exercise workload data for the individual; and a memory storing instructions that cause the processor to perform the above method.

A technical problem in the prior art is resolved, where the technical problem is the estimation of an individual's maximum oxygen uptake, VO2max, from exercise performed at a single exercise workload—such as at a single running speed.

Therefore, in contradistinction to the prior art, according to the method, the wearable device and the computing device for estimating an individual's maximum oxygen uptake, VO2max, of the disclosure, an individual's maximum oxygen uptake, VO2max, can be estimated from for example free-running exercises performed at a single speed, thus avoiding the discomfort caused to the individual by the requirement of running at different speeds. The method, the wearable device and the computing device according to the disclosure can improve the VO2max estimations by incorporating multiple exercise sessions and by taking account of the reliability of the measured data.

These and other aspects of the disclosure will be apparent from the implementations described below.

### BRIEF DESCRIPTION OF DRAWINGS

Implementations of the disclosure will now be described, by way of example only, with reference to the accompanying drawings, in which:

FIG. 1A is a block diagram of a wearable device to estimate an individual's maximum oxygen uptake, VO2max, during exercise in accordance with an implementation of the disclosure;

FIG. 1B is a block diagram of the wearable device coupled to a sensing arrangement in accordance with an implementation of the disclosure;

FIG. 1C is an exemplary view of the wearable device that is worn by an individual in accordance with an implementation of the disclosure;

FIG. 2 is a block diagram of a computing device to estimate an individual's maximum oxygen uptake, VO2max, during exercise in accordance with an implementation of the disclosure;

FIG. 3 is a process flow architecture of estimating an individual's maximum oxygen uptake, VO2max, during exercise in accordance with an implementation of the disclosure; and

FIG. 4 is a flow diagram that illustrates a method of estimating an individual's maximum oxygen uptake, VO2max, during exercise in accordance with an implementation of the disclosure.

### DETAILED DESCRIPTION OF THE DRAWINGS

Implementations of the disclosure provide a method of estimating an individual's maximum oxygen uptake, VO2max without the requirement for the user to perform exercise at multiple different rates or intensities, for example, from free-running exercises performed at a single running speed, or from a cycling exercise performed at a single exercise workload. Implementations of the disclosure provide a wearable device that is configured to estimate an individual's maximum oxygen uptake, VO2max without the requirement for the user to perform exercise at multiple different exercise workloads. Moreover, implementations of the disclosure provide a computing device that is configured to estimate an individual's maximum oxygen uptake, VO2max without the requirement for the user to perform exercise at multiple different exercise workloads.

To make solutions of the disclosure more comprehensible for a person skilled in the art, the following implementations of the disclosure are described with reference to the accompanying drawings.

In order to help understand implementations of the disclosure, several terms that will be introduced in the description of the implementations of the disclosure are defined herein first.

Terms such as “a first”, “a second”, “a third”, and “a fourth” (if any) in the summary, claims, and foregoing accompanying drawings of the disclosure are used to distinguish between similar objects and are not necessarily used to describe a specific sequence or order. It should be understood that the terms so used are interchangeable under appropriate circumstances, so that the implementations of the disclosure described herein are, for example, capable of being implemented in sequences other than the sequences illustrated or described herein. Furthermore, the terms “include” and “have” and any variations thereof, are intended to cover a non-exclusive inclusion. For example, a process, a method, a system, a product, or a device that includes a series of steps or units, is not necessarily limited to expressly listed steps or units but may include other steps or units that are not expressly listed or that are inherent to such process, method, product, or device.

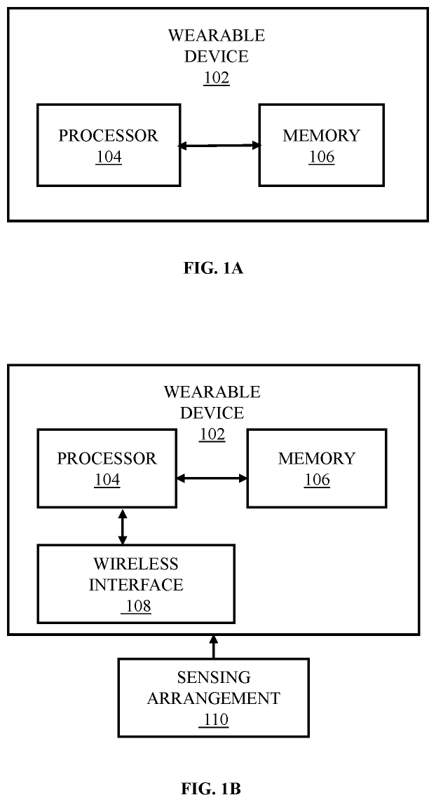

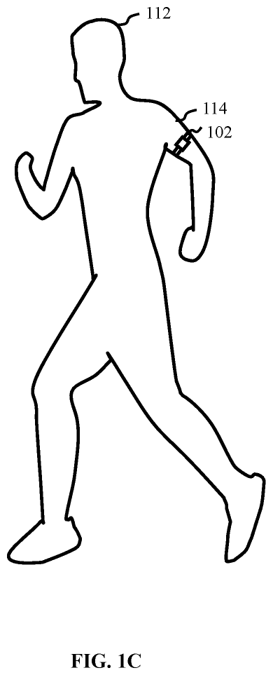

FIG. 1A is a block diagram of a wearable device 102 to estimate an individual's maximum oxygen uptake, VO2max, during exercise in accordance with an implementation of the disclosure. The wearable device 102 includes a processor 104 coupled to a memory 106. The processor 104 is configured to receive heart-rate measurement data and exercise workload data for an individual (e.g. a user) of the wearable device 102. The memory 106 is configured to store instructions that cause the processor 104 to perform the above method. The processor 104 is configured to normalise the obtained heart-rate with respect to the individual's maximum heart-rate to provide a data pair (or tuple) of normalised heart-rate, HRn, and exercise workload, w. The processor 104 is configured to apply a probabilistic model that relates normalised heart-rate to exercise workload and maximum oxygen uptake to provide an estimate of the individual's maximum oxygen uptake, VO2max.

The wearable device 102 estimates the individual's maximum oxygen uptake, VO2max, from, for example free-running exercises performed at a single running speed, thus avoiding the discomfort caused to the individual by, for example, the requirement to run at different speeds for estimating the maximum oxygen uptake, VO2max. The wearable device 102 determines the individual's maximum oxygen uptake, VO2max, in terms of a probability distribution from a proportion of maximum heart-rate and workload measurements—such as running speed measurements from free-running exercises which may be performed at the same speed. The wearable device 102 can further improve VO2max estimation by using data from multiple exercise sessions.

The exercise workload data may include global positioning system (GPS) data, speed data (e.g. running speed data), step-rate, cadence of the individual. The heart-rate measurement data may comprise the heart-rate that may or may not be averaged over a few heart-rate intervals. The wearable device 102 here optionally includes a heart-rate monitor for capturing heart-rate data from the user of the wearable device 102, but the wearable device 102 may instead be configured to receive heart rate data from an external sensing arrangement—either wirelessly or through a wired connection.

FIG. 1B is a block diagram of the wearable device 102 coupled to a sensing arrangement 110, which may be one of multiple sensing arrangements, in accordance with another implementation of the disclosure. The wearable device 102 is communicatively connected to the sensing arrangement 110. The wearable device 102 includes the processor 104 that is coupled to the memory 106. The wearable device 102 of FIG. 1B includes a wireless interface 108 for receiving the heart-rate measurement data and the exercise workload data from one or more sensing arrangement 110 external to the wearable device 102. The sensing arrangement 110 optionally measures the individual's heart-rate and running speed once every 5 seconds. The sensing arrangement 110 may optionally measure other physiological parameters of the individual while performing exercise. The sensing arrangement 110 may include, for example, a bicycle power meter—such as pedal or crank based power meter, to capture exercise workload data during a cycling session. The sensing arrangement 110 may include a GNSS receiver (such as a GPS receiver) to receive satellite navigation signals to enable the location, elevation, and velocity of a user of the device to be determined. The sensing arrangement 110 may additionally include one or more accelerometers to capture movement (e.g. step count and step rate) data, from which an individual's walking/running speed and distance travelled (using knowledge of stride length) may be determined.

FIG. 1C is an exemplary view of the wearable device 102 that is worn by an individual (i.e. a user) 112 in accordance with an implementation of the disclosure. The wearable device 102 is optionally worn by the individual 112 on his/her arm 114. The wearable device 102 may be comfortably worn at any location on the body of the individual 112 that allows estimation of the individual's maximum oxygen uptake, VO2max, during exercise. For example, the wearable device 102 may be worn on the user's chest, possibly integrated with a heart-rate sensor positioned over or adjacent the user's heart.

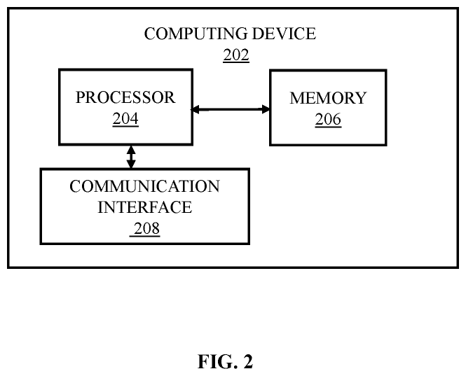

FIG. 2 is a block diagram of a computing device 202 to estimate an individual's maximum oxygen uptake, VO2max, during exercise in accordance with an implementation of the disclosure. The computing device 202 includes a processor 204, a memory 206, and a communication interface 208 coupled to the processor 204. The communication interface 208 receives heart-rate measurement data and exercise workload data for the individual—from an internal sensing arrangement, from an external sensing arrangement, or some combination of the two. The memory 206 is configured to store instructions that cause the processor 204 to perform any of the above described methods. The processor 204 receives heart rate and exercise workload data and is configured to normalise the obtained heart-rate with respect to the individual's maximum heart-rate to provide a data pair (or tuple) of normalised heart-rate, HRn, and exercise workload, w. The processor 204 is configured to apply a probabilistic model that relates normalised heart-rate to exercise workload and maximum oxygen uptake to provide an estimate of the individual's maximum oxygen uptake, VO2max.

The computing device 202 estimates the maximum oxygen uptake, VO2max of the individual in terms of a probability distribution from a proportion of maximum heart-rate and exercise workload measurements from, for example free-running exercises performed at the same speed. The computing device 202 can improve the VO2max estimations by utilising data from multiple exercise sessions and also by taking account of the reliability of the measured data. The computing device 202, without limitation, may be selected from a mobile phone, a smart watch, a Personal Digital Assistant (PDA), a tablet, a desktop computer, a server, or a laptop.

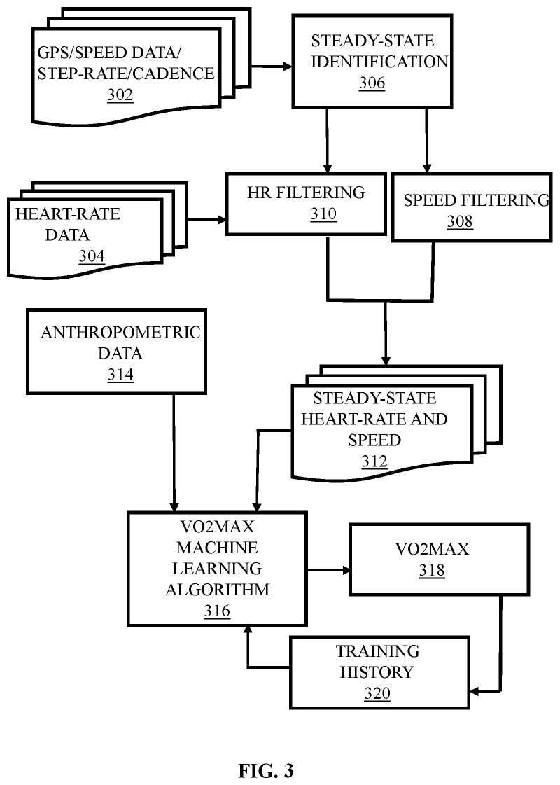

FIG. 3 is a process flow architecture of estimating an individual's maximum oxygen uptake, VO2max, during exercise in accordance with an implementation of the disclosure. At a step 302, a measurement of exercise workload data of the individual that includes speed data is obtained. The exercise workload data of the individual may include Global positioning system (GPS) data, power meter data (for example from a bicycle or stationary cycle power meter), step-rate (for example from an accelerometer of or associated with or part of a wearable device, or from a running machine), and/or cadence of the individual. At a step 304, heart-rate measurement data is obtained. At a step 306, a steady-state of the individual is identified using the exercise workload data (e.g. speed data, or power meter data). The steady-state of the individual may include running stability and constant motion of the individual while performing the exercise. At a step 308, the speed data of the individual are filtered to identify steady-state speed data. Filtering of the speed data may include discarding unstable data using a sliding window technique (thereby improving reliability). The sliding window technique determines a maximum speed variation in the measurements of the speed data by calculating the difference between the maximum and minimum speed within a sliding-window. At a step 310, the heart-rate measurement data is filtered corresponding to the steady-state speed data to obtain steady-state heart-rate data. The steady-state heart-rate may be calculated from the heart-rate measurements corresponding to the speed data measurements falling within the sliding-window. At a step 312, the steady-state heart-rate data and the steady-state speed data of the individual are obtained. At a step 314, anthropometric data of the individual are obtained. The anthropometric data may include measurement of dimensional descriptors (e.g. height, weight, leg length, body mass index, etc.) and physical properties (e.g. sex, and age) of the individual's body. At a step 316, a VO2max machine learning algorithm is applied. The VO2max machine learning algorithm employs a probabilistic model to calculate a probability distribution to determine the probability of each possible VO2max value using the steady-state heart-rate data and the corresponding workload data. At a step 318, the individual's maximum oxygen uptake, VO2max, is determined. At a step 320, the determined individual's maximum oxygen uptake, VO2max, is stored in order to improve the accuracy of estimated VO2max in future exercise sessions.

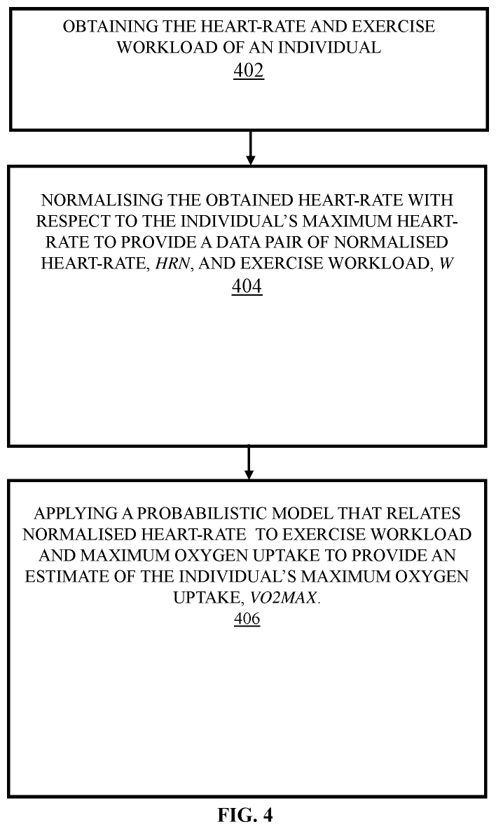

FIG. 4 is a flow diagram that illustrates a method for estimating an individual's maximum oxygen uptake, VO2max, during exercise in accordance with an implementation of the disclosure. The individual has a heart-rate. At a step 402, the heart-rate and exercise workload of the individual are obtained. At a step 404, the obtained heart-rate is normalized with respect to the individual's maximum heart-rate to provide a data pair (or tuple) of normalised heart-rate, HRn, and exercise workload, w. At a step 406, a probabilistic model that relates normalised heart-rate to exercise workload and maximum oxygen uptake is applied to provide an estimate of the individual's maximum oxygen uptake, VO2max. The method may include estimating the individual's maximum oxygen uptake, VO2max, from exercise undertaken at a single exercise workload—e.g. from free-running exercises performed at a single running speed. Additionally, the method can improve the VO2max estimations by incorporating multiple exercise sessions and taking account of the reliability of the measured data. The method optionally includes storing multiple data pairs (multiple tuples) of normalised heart-rate, HRn, and exercise workload, w determined periodically throughout an exercise session.

In a first implementation, the estimate of the individual's maximum oxygen uptake, VO2max, is provided by using the probabilistic model to determine a probability density function p(VO2max|HRn, w). The probability density function p(VO2max|HRn, w) is optionally determined using Bayes' Rule:

$$p ( VO 2 max \vert HRn , w ) = \frac{p ( HRn , w \vert VO 2 max ) p ( VO 2 max )}{p ( HRn , w )}$$

In a second implementation, the method includes storing the probability density function p(VO2max|HRn, w) after determining each data pair. The method may include storing the probability density function comprises discretizing the probability density function and storing the discretized values. The method may include discretizing the probability density function comprises calculating p(VO2max|HRn, w) for a set of discrete VO2max values, and storing the resulting values.

The method optionally includes using the last-stored probability density function pₜ₋₁(VO2max|HRnₜ₋₁, wₜ₋₁) in place of p(VO2max):

$$p ( VO 2 max \vert HRn , w ) = \frac{p ( HRn , w \vert VO 2 max ) {p}_{t - 1} ( VO 2 max \vert {HRn}_{t - 1} , {w}_{t - 1} )}{p ( HRn , w )}$$

The method optionally includes modifying the last-stored probability density function pₜ₋₁(VO2max|HRnₜ₋₁, wₜ₋₁) by increasing its uncertainty with respect to VO2max as a function of time since the last-stored probability density function pₜ₋₁(VO2max|HRnₜ₋₁, wₜ₋₁) was stored, denoted by [symbol] (VO2max|HRnₜ₋₁, w) and resulting in:

$$p ( VO 2 max \vert HRn , w ) = \frac{p ( HRn , w \vert VO 2 max ) ( VO 2 max \vert {HRn}_{t - 1} , {w}_{t - 1} )}{p ( HRn , w )}$$

Optionally, the uncertainty of the last-stored probability density function pₜ₋₁(VO2max|HRnₜ₋₁, wₜ₋₁) with respect to VO2max is only increased if the time since the last-stored probability density function pₜ₋₁(VO2max|HRnₜ₋₁, wₜ₋₁) was stored exceeds 1 day. Optionally, p(VO2max) relates one or more of the individual's age, gender, body-mass index, and physical activity level to VO2max.

The method may include determining the mean of the probability density function p(VO2max|HRn, w) to provide the estimate of the individual's maximum oxygen uptake. The method may include determining a VO2max value that maximizes the probability density function p(VO2max|HRn, w), to provide the estimate of the individual's maximum oxygen uptake.

The probabilistic model may be derived from a dataset containing multiple individuals exercise workload data, heart-rate data and VO2max obtained from cardiopulmonary exercise tests. The probabilistic model is optionally based on a multivariate Gaussian distribution.

The method may include identifying and discarding normalised heart-rate and exercise workload data pairs that lead to

p(HRn,w|VO2max)=0,∀VO2max

Measuring the exercise workload may be performed by determining a running speed of the individual during exercise. Optionally, measuring the exercise workload is performed using a bicycle power meter (for example, a pedal power meter or a crank power meter). Optionally, measuring the exercise workload is performed using a power meter of a stationary exercise machine, such as a rowing machine or a stationary bike.

The individual's maximum heart-rate may be estimated based on the individual's age. Optionally, the event that the individual's maximum measured heart-rate is determined to exceed the maximum heart-rate estimated based on the individual's age, the maximum measured heart-rate is used in place of the maximum heartrate estimated based on the individual's age. The individual's maximum heart-rate may be estimated based on heart-rate measurements obtained from the individual during exercise.

In an example implementation, the exercise workload, w, includes a running speed of the individual. The measurements of the running speed are used for assessing running stability and constant motion of the individual. Optionally, a sliding window with a fixed duration within the range of 60 to 120 seconds, for example 90 seconds, determines a maximum speed variation in the measurements of the running speed by calculating a difference between a maximum and a minimum running speed within the sliding window. The entire running speed measurements within the sliding window may be deemed unstable and discarded if the difference between the maximum and minimum running speed is larger than, for example, 1 kilometer per hour (km/h). New running speed measurements may be fed into the sliding-window if the entire speed measurements are unstable. Optionally, an average heart-rate is calculated from the heart-rate measurements corresponding to the running speed measurements falling within the sliding window and then a data pair (tuple) of average heart-rate and average running speed is obtained. This process may be repeated for the entire duration of the running exercise, for example, and in case multiple stable speeds are identified, a set of average heart-rate and average running speed data pairs (tuples) may be obtained by the end of the running exercise.

For each average heart-rate, a normalized average heart-rate is calculated by dividing the average heart-rate by an estimate of the maximum heart-rate of the individual. The estimate of the maximum heart-rate of the individual may be identified by using the expression 220—age, for example.

Then, from each normalized average heart-rate and average running speed data pair, a probability density function (PDF) for the VO2max is calculated by employing Bayes' rule:

$$p ( VO 2 max \vert HRn , v ) = \frac{p ( HRn , v \vert VO 2 max ) p ( VO 2 max )}{p ( HRn , v )}$$

Here, p (VO2max) denotes a probability density function (PDF) for the VO2max that is obtained before acquiring normalized heart-rate and running speed data pairs (i.e. measurement pair) (HRn, v). Anthropometric data of the individual may be used for determining p(VO2max). Alternatively, in case p (VO2max) is unknown, it can be set to 1. Optionally, the normalized heart-rate and running speed data pairs (HRn, v), that lead to p(HRn, v|VO2max)=0, ∀VO2max, are identified and discarded (thereby improving reliability).

Optionally, the sequential measurements of the heart-rate and the running speed are used directly without assessing running stability and constant motion of the individual, after normalization by the individual's maximum heart-rate.

Two measurement pairs (HRn₁, v₁) and (HRn₂, v₂) may be obtained at the end of the running exercise of the individual. The probability density function (PDF) for the VO2max with the first measurement pair (HRn₁, v₁) may be calculated by employing Bayes' rule as follows:

$$p ( VO 2 max \vert {HRn}_{1} , {v}_{1} ) = \frac{p ( {HRn}_{1} , {v}_{1} \vert VO 2 max )}{p ( {HRn}_{1} , {v}_{1} )}$$

The probability density function (PDF) for the VO2max with the second measurement pair (HRn₂, v₂) may be calculated by employing Bayes' rule as follows:

$$p ( VO 2 max \vert {HRn}_{2} , {v}_{2} ) = \frac{p ( {HRn}_{2} , {v}_{2} \vert VO 2 max ) p ( VO 2 max )}{p ( {HRn}_{2} , {v}_{2} )}$$

The PDF p(HRn₂, v₂) may be determined from as follows:

p(HRn₂,v₂)=∫p(HRn₂,v₂|VO2max)p(VO2max)dVO2max

The PDF p(HRn₂, v₂) may be approximated as a sum over a discretized set of VO2max values (for example, VO2maxᵢ∈(20 ml/kg/min, . . . , 90 ml/kg/min)), as follows:

$$p ( {HRn}_{2} , {v}_{2} ) = {\sum }_{VO 2 {max}_{i}} p ( {HRn}_{2} , {v}_{2} \vert VO 2 {max}_{i} ) p ( VO 2 {max}_{i} )$$

In an example implementation, a probabilistic model relating the normalized heart-rate p(HRn, v|VO2max) is determined using running speed, and VO2max The probabilistic model may be acquired from a dataset that includes individuals' running data and VO2max obtained from standard cardiopulmonary exercise tests. In particular, the following relationship between a joint PDF, a conditional PDF, and a marginal PDF may be used.

$$p ( HRn , v \vert VO 2 max ) = \frac{p ( HRn , v , VO 2 max )}{p ( VO 2 max )}$$

The dataset may be used to determine the probabilistic model relating the normalized heart-rate p(HRn, v, VO2max). For example, p(HRn, v, VO2max) may be given by a multivariate Gaussian distribution as follows:

$$p ( HRn , v , VO 2 max ) = \frac{1}{{( 2 π )}^{3 / 2}} \frac{1}{{\vert \sum \vert}^{1 / 2}} {e}^{- \frac{1}{2} {( {[ HRn , v , VO 2 max ]}^{T} - μ )}^{T} {\sum }^{- 1} ( {[ HRn , v , VO 2 max ]}^{T} - μ )}$$

Here, |Σ| denotes a determinant of the (3×3) covariance matrix Σ and μ denotes the (3×1) mean-vector of the distribution. Exact values for Σ and μ may be obtained from standard cardiopulmonary exercise tests by fitting p(HRn, v, VO2max) to such a dataset while maintaining its generalizability. In particular, Σ and μ may be given as follows (with realistic exemplary data values provided here):

$$μ = {[ 0.82 , 2.77 , 48.64 ]}^{T}$$

$$\sum = [ \begin{matrix}0.02 & 0.11 & 0.14 \\ 0.11 & 0.92 & 4.17 \\ 0.14 & 4.17 & 72.85\end{matrix} ]$$

In this case, it follows that p (VO2max) is also Gaussian with a mean and a variance given by 48.64 and 72.85, respectively. Then, the conditional PDF p(HRn, v|VO2max) is also Gaussian with a (2×1) mean-vector m and (2×2) covariance matrix C given by

$$m = {[ 0.82 , 2.77 ]}^{T} + {[ 0.0019 , 0.0572 ]}^{T} ( VO 2 max - 48.64 )$$

$$C = [ \begin{matrix}0.0217 & 0.102 \\ 0.102 & 0.6813\end{matrix} ]$$

Furthermore, the marginal PDF p(HRn, v) is Gaussian with a (2×1) mean-vector and (2×2) covariance matrix Q given by

$$ζ = {[ 0.82 , 2.77 ]}^{T}$$

$$Q = [ \begin{matrix}0.02 & 0.11 \\ 0.11 & 0.92\end{matrix} ]$$

Optionally, the probabilistic models for p(HRn, v, VO2max), p(HRn, v|VO2max), or p(VO2max) other than Gaussian distributions are accommodated.

The posterior PDF p(VO2max|HRn, v) may be determined numerically from the Bayes' rule given above, and its mean provides an estimate for the VO2max of the individual:

$$V = {\sum }_{VO 2 {max}_{i}} p ( VO 2 max \vert HRn , v ) VO 2 {max}_{i}$$

The posterior PDF is with respect to current measurement data pair or tuple. The posterior PDF describes a probability distribution of the VO2max of the individual after having observed a measurement tuple of the data pair of normalised heart-rate, HRn, and the exercise workload, w. The posterior PDF may be a prior PDF for a subsequent measurement tuple. The prior PDF describes information about the VO2max before a new measurement tuple is determined. The prior PDF is then adjusted based on the new measurement tuple. This process repeats for each measurement tuple (data pair). Alternatively, an estimate for the individual's VO2max can be obtained by determining the VO2max value that maximizes p(VO2max|HRn, v). Optionally, the posterior PDF p(VO2max|HRn, v) is updated and stored after each running exercise session, according to the Bayes' rule. At the beginning of each running exercise session, the posterior PDF p(VO2max|HRn, v) is used in place of p(VO2max).

It should be understood that the arrangement of components illustrated in the figures described are exemplary and that other arrangement may be possible. It should also be understood that the various system components (and means) defined by the claims, described below, and illustrated in the various block diagrams represent components in some systems configured according to the subject matter disclosed herein. For example, one or more of these system components (and means) may be realized, in whole or in part, by at least some of the components illustrated in the arrangements illustrated in the described figures.

In addition, while at least one of these components are implemented at least partially as an electronic hardware component, and therefore constitutes a machine, the other components may be implemented in software that when included in an execution environment constitutes a machine, hardware, or a combination of software and hardware.

Although the disclosure and its advantages have been described in detail, it should be understood that various changes, substitutions, and alterations can be made herein without departing from the spirit and scope of the disclosure as defined by the appended claims.

1. A method comprising:
obtaining, during exercise, a heart-rate and exercise workload of an individual;
normalizing the heart-rate with respect to a maximum heart-rate of the individual to provide a data pair of normalized heart-rate (HRn) and the exercise workload (w); and
applying a probabilistic model that relates HRn to w and maximum oxygen uptake to provide an estimate of a maximum oxygen uptake of the individual (VO2max).

2. The method of claim 1, further comprising:
periodically determining, throughout an exercise session, multiple data pairs of HRn and w; and
storing the multiple data pairs of HRn and w.

3. The method of claim 1, further comprising determining, using the probabilistic model, a probability density function p(VO2max|HRn, w) to obtain the estimate of VO2max.

4. The method of claim 3, further comprising determining the probability density function p(VO2max|HRn, w) using Bayes' Rule, wherein the Bayes' Rule is:
$$p ( VO 2 max \vert HRn , w ) = \frac{p ( HRn , w \vert VO 2 max ) p ( VO 2 max )}{p ( HRn , w )}$$

5. The method of claim 2, further comprising storing a probability density function p(VO2max|HRn, w) after determining each of the multiple data pairs.

6. The method of claim 5, wherein storing the probability density function comprises:
discretizing the probability density function to obtain discretized values; and
storing the discretized values.

7. The method of claim 6, wherein discretizing the probability density function comprises:
calculating p(VO2max|HRn, w) for a set of discrete VO2max values to obtain resulting values; and
storing the resulting values.

8.-10. (canceled)

11. The method of claim 3, wherein p(VO2max) relates one or more of an age of the individual, a gender of the individual, a body-mass index of the individual, or a physical activity level of the individual to VO2max.

12. The method of claim 2, further comprising determining a mean of a probability density function p(VO2max|HRn, w) to provide the estimate of VO2max.

13. The method of claim 2, further comprising determining a value of the VO2max that maximizes a probability density function p(VO2max|HRn, w) to provide the estimate of VO2max.

14. The method of claim 1, further comprising deriving the probabilistic model from a dataset containing an exercise workload data of the individual, heart-rate data of the individual, and VO2max obtained from cardiopulmonary exercise tests.

15. The method of claim 1, wherein the probabilistic model is based on a multivariate Gaussian distribution.

16. The method of claim 1, further comprising identifying and discarding normalized heart-rate and exercise workload data pairs that lead to p(HRn, w|VO2max)=0, ∀VO2max.

17. The method of claim 1, further comprising measuring, by determining a running speed of the individual during exercise, the exercise workload.

18. The method of claim 1, further comprising measuring, using a bicycle power meter, the exercise workload.

19. The method of claim 1, further comprising measuring, using a power meter of a stationary exercise machine, the exercise workload, wherein the stationary exercise machine is a rowing machine or a stationary bike.

20. The method of claim 1, wherein further comprising estimating the maximum heart-rate estimated based on an age of the individual.

21. The method of claim 20, further comprising:
determining that a maximum measured heart-rate exceeds the maximum heart-rate that is estimated based on the age; and
setting, in response to determining that the maximum measured h rate exceeds the maximum heart-rate that is estimated based on the age, the maximum measured heart-rate as the maximum heart-rate.

22. (canceled)

23. A wearable device comprising:
a memory configured to store instructions; and
a processor coupled to the memory and configured to execute the instructions to cause the wearable device to:
receive heart-rate measurement data and exercise workload data for a user of the wearable device;
normalize the heart-rate measurement data with respect to a maximum heart-rate of the user to provide a data pair of normalized heart-rate (HRn) and exercise workload (w); and
apply a probabilistic model that relates HRn to w and maximum oxygen uptake to provide an estimate of a maximum oxygen uptake of the user (VO2max).

24.-25. (canceled)

26. A computer program product comprising computer-executable instructions that are stored on a non-transitory computer-readable medium and that when executed by a processor, cause a wearable device to:
receive heart-rate measurement data and exercise workload data for a user of the wearable device;
normalize the heart-rate measurement data with respect to a maximum heart-rate of the user to provide a data pair of normalized heart-rate (HRn) and exercise workload (w); and
apply a probabilistic model that relates HRn to w and maximum oxygen uptake to provide an estimate of a maximum oxygen uptake of the user (VO2max).
